(19) United States
(12) Patent Application Publication
(10) Pub. No.: US 2019/0062337 A1
(43) Pub. Date: Feb. 28, 2019

(54) PROCESSES AND INTERMEDIATES FOR THE PREPARATION OF {1-(ETHYLSULFONYL)-3-[4-(7H-PYRROLO[2,3-d]PYRIMIDIN-4-YL)-1H-PYRAZOL-1-YL]AZETIDIN-3-YL}ACETONITRILE

(71) Applicant: Eli Lilly and Company, Indianapolis, IN (US)

(72) Inventors: Michael Edward Kobierski, Greenwood, IN (US); Michael E. Kopach, Greenwood, IN (US); Joseph R. Martinelli, Indianapolis, IN (US); David Lee Varie, Indianapolis, IN (US); Thomas Michael Wilson, Speedway, IN (US)

(21) Appl. No.: 16/170,137

(22) Filed: Oct. 25, 2018

Related U.S. Application Data
(63) Continuation of application No. 15/579,612, filed on Dec. 5, 2017, now abandoned.
(60) Provisional application No. 62/182,040, filed on Jun. 19, 2015.

(51) Int. Cl. C07D 487/04; C07F 5/02; A61K 31/519

(52) U.S. Cl. CPC C07D 487/04; C07F 5/025; A61K 31/519

(57) ABSTRACT

The present invention provides processes and intermediates for the preparation of {1-(ethylsulfonyl)-3-[4-(7H-pyrrolo[2,3-d]pyrimidin-4-yl)-1H-pyrazol-1-yl]azetidin-3-yl}acetonitrile: (I)

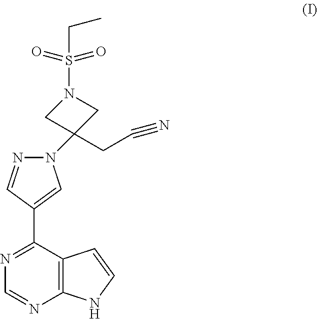 [SMILES: CCS(=O)(=O)N1CC(CC#N)(n2cc(-c3ncnc4[nH]ccc34)cn2)C1]

The present invention relates to the fields of pharmaceutical chemistry and synthetic organic chemistry, and provides processes and key intermediates for the synthesis of {1-(ethylsulfonyl)-3-[4-(7H-pyrrolo[2,3-d]pyrimidin-4-yl)-1H-pyrazol-1-yl]azetidin-3-yl}acetonitrile, a JAK1 and JAK2 inhibitor.

Janus kinase-1 (JAK1) and Janus kinase-2 (JAK2) are two members of the Janus Kinase (JAK) family which play a role in the cytokine-dependent regulation of proliferation and function of cells involved in immune response. Blocking signal transduction at the level of the JAK kinases holds promise for developing treatments for diseases, such as inflammatory diseases, autoimmune diseases, myeloproliferative diseases, and human cancers. Baricitinib, {1-(ethylsulfonyl)-3-[4-(7H-pyrrolo[2,3-d]pyrimidin-4-yl)-1H-pyrazol-1-yl]azetidin-3-yl}acetonitrile, illustrated as (I) below, is an inhibitor of JAK1 and JAK2 and is taught to be useful for treating inflammatory diseases, such as rheumatoid arthritis. See WO 2009/114512.

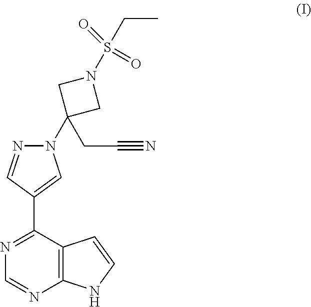 [SMILES: CCS(=O)(=O)N1CC(CC#N)(n2cc(-c3ncnc4[nH]ccc34)cn2)C1]

The present invention provides a process for the preparation of {1-(ethylsulfonyl)-3-[4-(7H-pyrrolo[2,3-d]pyrinaidin-4-yl)-1H-pyrazol-1-yl]azetidin-3-yl}acetonitrile (I), comprising the steps of:
i) Coupling azetidine-3-ol hydrochloride (2) with ethanesulfonyl chloride to give 1-ethylsulfonylazetidin-3-ol (3);
ii) Aerobic oxidation of 1-ethylsulfonylazetidin-3-ol (3) to 1-(ethylsulfonyl)azetidin-3-one (4) in the presence of a nitroxyl reagent, an oxidizing reagent, and an acid under an oxygen atmosphere under flow or batch conditions; Alternatively, oxidation of 1-ethylsulfonylazetidin-3-ol (3) to 1-(ethylsulfonyl)azetidin-3-one (4) with TCCA and a catalytic oxammonium reagent under batch conditions;
iii) Reaction of 1-(ethylsulfonyl)azetidin-3-one (4) with a phosphonate reagent in the presence of a base to prepare compound (1);
iv) Optionally crystallizing [1-(ethylsulfonyl)azetidin-3-ylidene]acetonitrile (1);
v) Optionally protecting 4-(4,4,5,5-tetramethyl-1,3,2-dioxaborolan-2-yl)-1H-pyrazole (5) with a nitrogen protecting group;
vi) Coupling [1-(ethylsulfonyl)azetidin-3-ylidene]acetonitrile(1) and 4-(4,4,5,5-tetramethyl-1,3,2-dioxaborolan-2-yl)-1H-pyrazole (5) in the presence of a non-nucleophilic base to give (II);
vii) Optionally crystallizing {1-(ethylsulfonyl)-3-[4-(4,4,5,5-tetramethyl-1,3,2-dioxaborolan-2-yl)-1H-pyrazol-1-yl]azetidin-3-yl}acetonitrile (II);
viii) Optionally protecting 4-chloro-7H-pyrrolo[2,3-d]pyrimidine (7a) with a nitrogen protecting group;
ix) Coupling {1-(ethylsulfonyl)-3-[4-(4,4,5,5-tetramethyl-1,3,2-dioxaborolan-2-yl)-1H-pyrazol-1-yl]azetidin-3-yl}acetonitrile (II) with 4-chloro-7H-pyrrolo[2,3-d]pyrimidine (7a) or tert-butyl 4-chloropyrrolo[2,3-d]pyrimidine-7-carboxylate (7b) using a Pd(II) catalyst in the presence of a base to provide {1-(ethylsulfonyl)-3-[4-(7H-pyrrolo[2,3-d]pyrimidin-4-yl)-1H-pyrazol-1-yl]azetidin-3-yl}acetonitrile (I) or tert-butyl 4-{1-[3-(cyanomethyl)-1-(ethylsulfonyl)azetidin-3-yl]-1H-pyrazol-4-yl}-7H-pyrrolo[2,3-d]pyrimidine-7-carboxylate (III);
x) Optionally deprotecting tert-butyl 4-{1-[3-(cyanomethyl)-1-(ethylsulfonyl)azetidin-3-yl]-1H-pyrazol-4-yl}-7H-pyrrolo[2,3-d]pyrimidine-7-carboxylate (III) to {1-(ethylsulfonyl)-3-[4-(7H-pyrrolo[2,3-d]pyrimidin-4-yl)-1H-pyrazol-1-yl]azetidin-3-yl}acetonitrile (I); and
xi) Optionally crystallizing {1-(ethylsulfonyl)-3-[4-(7H-pyrrolo[2,3-d]pyrimidin-4-yl)-1H-pyrazol-1-yl]azetidin-3-yl}acetonitile (I).

In a further embodiment of the present invention, the nitroxyl reagent of step ii) is TEMPO, 4-AATEMPO, 4-hydroxyTEMPO, 3-carbamoyl-PROXYL, AZADO, or ABNO. In another further embodiment of the present invention, the nitroxyl reagent may be used in an amount of <1-100%. In yet a further embodiment, the amount of nitroxyl reagent is 5%. In another further embodiment of the present invention, the oxidizing reagent of step ii) is NaNO₂. In a further embodiment of the present invention, the acid of step ii) is acetic acid or nitric acid. In yet a further embodiment, the preferred acid of step ii) is acetic acid. In yet another embodiment of the present invention, the % oxygen of the reaction for step ii) is <1% up to the just under the LOC (limiting oxygen concentration) for the given solvent being used, however it is preferable to operate in a range of 5 to 8% O₂ in N₂. In yet a further embodiment of the present invention, the oxygen atmosphere of step ii) is 6% O₂ in N₂. In yet another further embodiment of the present invention, the oxygen atmosphere of step ii) is 8% O₂ in N₂. In yet another further embodiment of the present invention, the phosphonate reagent of step ii) is diethyl cyanomethylphosphonate.

Alternatively, NaOCl (bleach), Br₂, or Ph1(OAc)₂ may be used as the oxidizing reagent of step ii) in place of NaNO₂ without the addition of an acid or addition of oxygen to the reaction atmosphere.

In a further embodiment of the present invention of the step ii) under alternative oxidation conditions, the oxidation is run optimally with TCCA and a catalytic amount of oxammonium TEMPO, HOT, 4AA TEMPO, AZADO or 3-carbamoyl-PROXYL at 1000 or less substrate to catalyst (S/C) ratios. A key component of the invention is pre mixing the oxammonium catalyst with substrate which allows for maximization of catalytic activity. In yet another further embodiment, the preferred acid of step ii) is TCCA when performed using batch processing methodology.

In yet another further embodiment of the present invention, the phosphonate reagent of step iii) is diethyl cyanomethylphosphonate.

In a further embodiment of the present invention, the base of step iii) is DIPEA.

In a further embodiment of the present invention the nitrogen protecting group of step v) is Boc, THP, FMOC, TIPS, ethoxyethyl, or methoxyethyl. In yet a further embodiment, the nitrogen protecting group is ethoxyethyl or methoxyethyl. In yet another further embodiment, the nitrogen protection group is ethoxyethyl.

In another further embodiment of the present invention, the base of step vi) is DBU, 2-tert-butyl-1,1,3,3-tetramethylguanidine, potassium tert-butoxide, or tetramethylguanidine. In yet another preferred embodiment, the base is 2-tert-butyl-1,1,3,3-tetramethylguanidine.

In a further embodiment of the present invention, the nitrogen protecting group of step viii) is Boc, THP, ethoxy ethyl, CBZ.

In another further embodiment of the present invention, the Pd(II) catalyst of step ix) is dichloro[1,1′-bis(dicyclohexylphosphino)ferrocene] palladium(II), PdCl₂-XantPhos, DPPF, or PdCl₂(dtbpf).

In yet a further embodiment of the present invention, the base of step ix) is K₃PO₄, potassium tert-butoxide, sodium carbonate, or sodium bicarbonate.

In yet another further embodiment of the present invention, the reaction may be performed in a biphasic reaction mixture of organic and aqueous solvents. In a further embodiment of the present invention, the reaction may be performed in THF with an aqueous solution which is basic. In a further embodiment of the present invention, the product of each step of the process is isolated. In yet a further embodiment, the product of each step is not isolated but carried on directly to the next step.

The present invention also provides a process for the preparation of {1-(ethylsulfonyl)-3-[4-(7H-pyrrolo[2,3-d]pyrimidin-4-yl)-1H-pyrazol-1-yl]azetidin-3-yl}acetonitrile (I), comprising the steps of:
viii) Optionally protecting 4-chloro-7H-pyrrolo[2,3-d]pyrimidine (7a) with a nitrogen protecting group;
ix) Coupling {1-(ethylsulfonyl)-3-[4-(4,4,5,5-tetramethyl-1,3,2-dioxaborolan-2-yl)-1H-pyrazol-1-yl]azetidin-3-yl}acetonitrile (II) with 4-chloro-7H-pyrrolo[2,3-d]pyrimidine (7a) or tert-butyl 4-chloropyrrolo[2,3-d]pyrimidine-7-carboxylate (7b) using a Pd(II) catalyst in the presence of a base to provide {1-(ethylsulfonyl)-3-[4-(7H-pyrrolo[2,3-d]pyrimidin-4-yl)-1H-pyrazol-1-yl]azetidin-3-yl}acetonitrile (I) or tert-butyl 4-{1-[3-(cyanomethyl)-1-(ethylsulfonyl)azetidin-3-yl]-1H-pyrazol-4-yl}-7H-pyrrolo[2,3-d]pyrimidine-7-carboxylate (III);
x) Optionally deprotecting tert-butyl 4-{1-[3-(cyanomethyl)-1-(ethylsulfonyl)azetidin-3-yl]-1H-pyrazol-4-yl). -7H-pyrrolo[2,3-d]pyrimidine-7-carboxylate (III) to {1-(ethylsulfonyl)-3-[4-(7H-pyrrolo[2,3-d]pyrimidin-4-yl)-1H-pyrazol-1-yl]azetidin-3-yl}acetonitrile (I); and
xi) Optionally crystallizing {1-(ethylsulfonyl)-3-[4-(7H-pyrrolo[2,3-d]pyrimidin-4-yl)-1H-pyrazol-1-yl]azetidin-3-yl)acetonitrile (I).

In a further embodiment of the present invention, the nitrogen protecting group of step viii) is Boc. In another further embodiment of the present invention, the Pd(II) catalyst of step ix) is dichloro[1,1′-bis(dicyclohexylphosphino)ferrocene] palladium(II), PdCl₂-XantPhos, DPPF, or PdCl₂(dtbpf). In yet a further embodiment of the present invention, the base of step ix) is K₃HPO₄, potassium tert-butoxide, sodium carbonate, or sodium bicarbonate. In yet another further embodiment of the present invention, the reaction may be performed in a biphasic reaction mixture of organic and aqueous solvents. In a further embodiment of the present invention, the reaction may be performed in THF with an aqueous solution which is basic. In a further embodiment of the present invention, the product of each step of the process is isolated. In yet a further embodiment, the product of each step is not isolated but carried on directly to the next step.

The present invention also provides a process for the preparation of 2-[1-ethylsulfonyl-3-[4-(4,4,5,5-tetramethyl-1,3,2-dioxaborolan-2-yl)pyrazol-1-yl]azetidin-3-yl]acetonitrile (II) comprising the steps of:
vi) Coupling (1) and (5) in the presence of a non-nucleophilic base; and
vii) Optionally crystallizing 2-[1-ethylsulfonyl-3-[4-(4,4,5,5-tetramethyl-1,3,2-dioxaborolan-2-yl)pyrazol-1-yl]azetidin-3-yl]acetonitrile (II).

In a further embodiment of the present invention, the base of step v) is DKr, 2-tert-butyl-1,1,3,3-tetramethylguanidine, potassium tert-butoxide, or tetramethylguanidine. In another further embodiment, the base is 2-cert-butyl-1,1,3,3-tetramethylguanidine. In a further embodiment of the present invention, the product of each step of the process is isolated. In yet a further embodiment, the product of each step is not isolated but carried on directly to the next step.

The present invention also provides a process for the preparation of [1-(ethylsulfonyl)azetidin-3-ylidene]acetonitrile (1) comprising the steps of:
i) Coupling azetidine-3-ol hydrochloride (2) with ethanesulfonyl chloride to give 1-ethylsulfonylazetidin-3-ol (3);
ii) Aerobic oxidation of the alcohol of 1-ethylsulfonylazetidin-3-ol (3) to 1-(ethylsulfonyl)azetidin-3-one (4) in the presence of a nitroxyl reagent, an oxidizing reagent, and an acidacid under an oxygen atmosphere under flow or batch conditions; Alternatively, oxidation of 1-ethylsulfonylazetidin-3-ol (3) to 1-(ethylsulfonyl)azetidin-3-one (4) with TCCA and a catalytic oxammonium reagent under batch conditions;
iii) Reaction of 1-(ethylsulfonyl)azetidin-3-one (4) with phosphonate reagent in the presence of a base to prepare compound (1); and
iv) Optionally crystallizing [1-(ethylsulfonyl)azetidin-3-ylidene]acetonitrile (1).

In a further embodiment of the present invention, the nitroxyl reagent of step ii) is TEMPO, 4-AATEMPO, 4-hydroxyTEMPO, 3-carbamoyl-PROXYL, AZADO, or ABNO. In another further embodiment of the present invention, the nitroxyl reagent may be used in an amount of <1%100%. In yet a further embodiment, the amount of nitroxyl reagent is 5%. In another further embodiment of the present invention, the oxidizing reagent of step ii) is NaNO₂. In a further embodiment of the present invention, the acid of step ii) is acetic acid or nitric acid. In yet a further embodiment, the preferred acid of step ii) is acetic acid. In yet another embodiment of the present invention, the % oxygen of the reaction for step ii) is <1% up to the just under the LOC (limiting oxygen concentration) for the given solvent being used, however it is preferable to operate in a range of 5 to 8% O₂ in N₂. In yet a further embodiment of the present invention, the oxygen atmosphere of step ii) is 6% O₂ in N₂. In yet another further embodiment of the present invention, the oxygen atmosphere of step ii) is 8% O₂ in N₂. In yet another further embodiment of the present invention, the phosphonate reagent of step ii) is diethyl cyanomethylphosphonate.

Alternatively, NaOCl (bleach), Br₂, or PhI(OAc)₂ may be used as the oxidizing reagent of step ii) in place of NaNO₂ without the addition of an acid or addition of oxygen to the reaction atmosphere.

In a further embodiment of the present invention of the step ii) under alternative oxidation conditions, the oxidation is run optimally with TCCA and a catalytic amount of oxammonium TEMPO, HOT, 4AA TEMPO, AZADO or 3-carbamoyl-PROXYL at 1000 or less substrate to catalyst (S/C) ratios. A key component of the invention is pre mixing the oxammonium catalyst with substrate which allows for maximization of catalytic activity. In yet another further embodiment, the preferred acid of step ii) is TCCA when performed using batch processing methodology.

In yet another further embodiment of the present invention, the phosphonate reagent of step iii) is diethyl cyanomethylphosphortate.

In a further embodiment of the present invention, the base of step iii) is DIPEA.

In a further embodiment of the present invention the product of each step of the process is isolated. In yet a further embodiment, the product of each step is not isolated but carried on directly to the next step.

An especially preferred embodiment of the present invention relates to the compound, 2-[1-ethylsulfonyl-3-[4-(4,4,5,5-tetramethyl-1,3,2-dioxaborolan-2-yppyrazol-1-yl]azetidin-3-yl]acetonitrile:

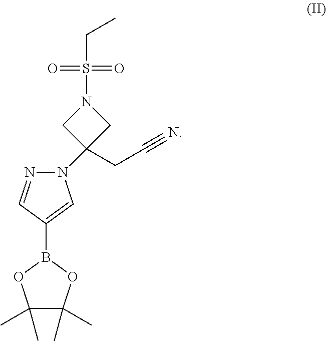 [SMILES: CCS(=O)(=O)N1CC(CC#N)(n2cc(B3OC(C)(C)C(C)(C)O3)cn2)C1]

A further especially preferred embodiment of the present invention provides a method of utilizing 2-[1-ethylsulfonyl-3-[4-(4,4,5,5-tetramethyl-1,3,2-dioxaborolan-2-yl)pyrazol-1-yl]azetidin-3-yl]acetonitrile (II) to prepare {1-(ethylsulfonyl)-3-[4-(7H-pyrrolo[2,3-d]pyrimidin-4-yl)-1H-pyrazol-1-yl]azetidin-3-yl}acetonitrile (I).

The terms “nitroxyl reagent” and “oxammonium reagent” can be used interchangeably.

The reactions described herein may be performed via standard techniques known to the skilled artisan by employing routine glassware but also by using autoclave pressure chambers. These reactions also may be performed on pilot and/or production scale in equipment designed for such transformations. Further, each of these reactions described may be executed via either a batch process or flow reaction methodology. The term “batch process” as used herein refers to a process in which raw materials are combined in a reactor or vessel and product is removed at the end of the reaction. The term “continuous processing” or “flow reaction” as used herein refers to a process in which there is a continuous inflow of raw materials and outflow of product. Such continuous processing enables a platform where the final product may be synthesized by a fully continuous train of operations starting from initial starting materials.

Individual isomers, enantiomers, and diastereomers may be separated or resolved by one of ordinary skill in the art at any convenient point in the synthesis of compounds of Formula I by methods such as selective crystallization techniques or chiral chromatography (See for example, J. Jacques, et al., “Enantiomers, Racemates, and Resolutions”, John Wiley and Sons, Inc., 1981, and E. L. Eliel and S. H. Wilen,” Stereochemistry of Organic Compounds“, Wiley-Interscience, 1994).

Additionally, certain intermediates described in the following preparations may contain one or more nitrogen protecting groups. The variable protecting group may be the same or different in each occurrence depending on the particular reaction conditions and the particular transformations to be performed. The protection and deprotection conditions are well known to the skilled artisan and are described in the literature (See for example “Greene's Protective Groups in Organic Synthesis”, Fourth Edition, by Peter G. M. Wuts and Theodora W. Greene, John Wiley and Sons, Inc. 2007).

The abbreviations used herein are defined as follows: “4-AA TEMPO” refers to 4-acetamido-(2,2,6,6-tetramethylpiperidin-1-yl)oxyl; “ABNO” refers to 9-azabicyclo[3.3.1]nonane N-oxyl; “Ac” refers to acetyl; “AGN” refers to acetonitrile; “AZADO” refers to 2-azaadamantane N-oxyl; “Boc” refers to tert-butyloxycarbonyl; “CBZ” refers to carboxybenzyl; “CPME” refers to cyclopentyl methylether; “CSTR” refers to continuous stirred tank reactor; “DBU” refers to 1,8-diazabicyclo[5.4.0]undec-7-ene; “DIPEA” refers to diisopropylethylamine; “DMF” refers to dimethylformamide; “DMSO” refers to dimethyl sulfoxide; “DPPF” refers to 1,1′-ferrocenediyl-bis(diphenylphosphino); “EtOAc” refers to ethyl acetate; “FMOC” refers to fluorenylmethyloxycarbonyl; “GC” refers to gas chromatography; “HPLC” refers to high-performance liquid chromatography; “IPA” refers to isopropanol or isopropyl alcohol; “KetoABNO” refers to 3-oxo-9-azabicyclo(3.3.1)non-9-yloxy; “LC/MS” refers to liquid chromatography-mass spectrometry; “2-MeTHF” refers to 2-methyl tetrahydrofuran; “MTBE” refers to methyl tert-butyl ether; “nor-AZADO” refers to 9-azanoradamantane N-oxyl; “PdCl₂(dtbpf)” refers to [1,1′-bis(di-tert-butylphosphino)ferrocene]dichloropalladium(II); “PdCl₂-XantPhos” refers to (9,9-dimethyl-9H-xanthene-4,5-diyl)bis(diphenylphosphane) dichloropalladium; “PhI(OAc)₂” refers to (diacetoxyiodo)benzene; “3-carbamoyl-PROXYL” refers to 3-carbamoyl-2,2,5,5-tetramethylpyrrolidinooxyl; “RAMAN” refers to Raman spectroscopy; “rpm” refers to revolutions per minute; “TCCA” refers to trichlorocyanuric acid or trichloroisocyanuric acid; “TEMPO” refers to 2,2,6,6-tetramethyl-1-piperidinyloxy free radical; “THF” refers to tetrahydrofuran; “THP” refers to tetrahydropyran; “TIPS” refers to triisopropylsilyl ether; and “TLC” refers to thin layer chromatography.

The compounds, or salts thereof, prepared by the synthesis described herein may be prepared by a variety of procedures known in the art, some of which are illustrated in the Schemes, Preparations, and Examples below. The specific synthetic steps for each of the routes described may be combined in different ways, or in conjunction with steps from different schemes. The products of each step in the schemes below can be recovered by conventional methods well known in the art, including extraction, evaporation, precipitation, chromatography, filtration, trituration, and crystallization. The reagents and starting materials are readily available to one of ordinary skill in the art. Reactions are typically followed to completion using techniques known to the skilled artisan, for example TLC, HPLC, GC, LC/MS, RAMAN, and the like. The skilled artisan will appreciate that the technique used will depend on a variety of factors including the scale of the reaction, the type of vessel in which the reaction is performed, and the reaction itself.

Compound (I), {1-(ethylsulfonyl)-3-[4-(7H-pyrrolo[2,3-d]pyrimidin-4-yl)-1H-pyrazol-1-yl]azetidin-3-yl }acetonitrile, is prepared beginning with 2-[1-ethylsulfonyl-3-[4-(4,4,5,5-tetramethyl-1,3,2-dioxaborolan-2-yl)pyrazol-1-yl]azetidin-3-yl]acetonitrile (II), illustrated in Scheme III. Compound (II), 2-[1-ethylsolfonyl-3-[4-(4,4,5,5-tetramethyl-1,3,2-dioxaborolan-2-yl)pyrazol-1-yl]azetidin-3-yl}acetonitrile, is prepared with 2-(1-ethylsulfonylazetidin-3-ylidene)acetonitrile (1) and 4-(4,4,5,5-tetramethyl-1,3,2-dioxaborolan-2-yl)-1H-pyrazole (5) by the procedure illustrated in Scheme II. Schemes I and II describe the synthesis of 2-(1-ethylsulfonylazetidin-3-ylidene)acetonitrile (1) and {1-(ethylsulfonyl)-3-[4-(4,4,5,5-tetramethyl-1,3,2-dioxaborolan-2-yl)-1H-pyrazol-1-yl]azetidin-3-yl}acetonitrile (II).

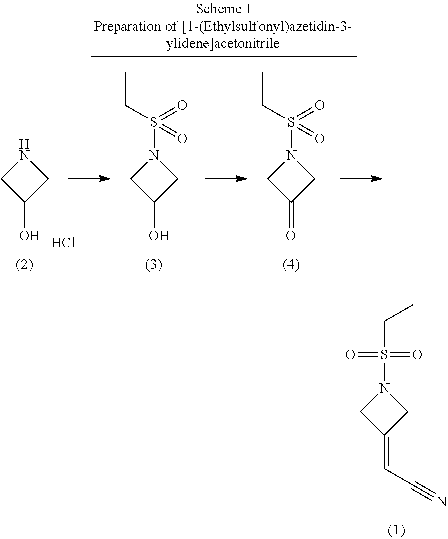

[1-(Ethylsulfonyl)azetidin-3-yliderie]acetonitrile (1) is synthesized by first treating azetidine-3-ol hydrochloride (2) with an equimolar equivalent of an alkanesulfonyl chloride, preferably ethanesulfonyl chloride, to give 1-ethylsulfonylazetidin-3-ol (3). Preferably, the reaction is performed in a biphasic solution comprising a mixture of an organic phase and an aqueous phase, preferably THF with an aqueous solution which is basic, while maintaining the solution at room temperature or a temperature slightly below room temperature, preferably 20° C. The reaction is followed to completion using standard monitoring techniques. Typically, the reaction is complete within 1 to 5 hours. The organic layer is removed, preferably by distillation, and the aqueous layer is extracted with an appropriate solvent such as toluene, p-cymene, and CPME, Preferably the extraction solvent is toluene. Alternatively, the toluene extractions can be excluded if recrystallization of (1) is performed. The aqueous layer is then extracted with an appropriate solvent, such as EtOAc, MTBE, and isopropylacetate, to give compound (3). Preferably, EtOAc is used to extract the aqueous layer. The compound may be isolated by standard techniques or taken on without further purification.

Alternatively, continuous counter current extraction may be used to isolate compound (3) using continuous extraction and settling operations linked together. A series of vessels such as continuously stirred tanks (CSTRs) can be used in combination with liquid-liquid separators to continuously extract species into or out of the desired phase. For example, the crude reaction mixture, after removal of the reaction solvent by distillation or other removal method, can be mixed with an appropriate solvent, such as toluene in one tank, then the phases can be separated in a liquid-liquid separator, and the resulting aqueous phase can be retreated with the appropriate solvent in this fashion as many times as required until the desired level of removal is achieved. The resulting aqueous phase can then be treated in the same way with an appropriate solvent, such as ethyl acetate to extract the product (3).

1-(Ethylsulfonyl)azetidin-3-one (4) is prepared by treating 1-(ethylsulfonyl)azetidin-3-ol (3) with a nitroxyl reagent, such as TEMPO, 4-hydroxyTEMPO, 4-acetamidoTEMPO, ABNO, PROXYL, 2-azaadamantane N-oxyl, KetoABNO, nor-AZADO, nortropane-N-oxyl, an oxidizing agent, for example sodium nitrite and an acid, such as acetic acid or nitric acid, in an appropriate solvent, such as water, acetonitrile, EtOAc, isopropyl acetate or other nitrile solvents or a mixture thereof and pressurized from about 14 psi to about 1000 psi, preferably about 500 psi, with a mixture of 5 to 8% O₂ in N₂, preferably 6% O₂ in N₂. The reagents may be added together at once or taken up in the appropriate solvent and added sequentially. Suitable nitroxyl reagents are described in ACS Catal. 2013, 3, 2612-2616 and Central Science, 2015, 1(5), 234-243. The preferred nitroxyl reagent is TEMPO. The preferred oxidizing agent is sodium nitrite. The preferred acid used in this reaction is acetic acid when performed using flow or batch reaction methodology. The temperature of the reaction may be held at room temperature or at a temperature above or lower than room temperature, preferably greater than 0° C. but less than 45° C. Optionally, the headspace of the reaction may be vented and replenished with the mixture of O₂ in N₂ every 60 to 600 seconds. Headspace recycling is important when the aerobic oxidation is run using batch process methodology and is not needed when run using flow reaction methodology. Typically the reaction is continued for 1-24 hours. Completion of the reaction is monitored by standard techniques known to the skilled artisan. The reaction product may be isolated by techniques known to the skilled artisan or taken on to the next reaction without isolation.

Alternatively, NaOCl (bleach), Br₂, or PhI(OAc)₂ may be used as the oxidizing reagent in place of NaNO₂ without the addition of an acid or addition of oxygen to the reaction atmosphere.

Alternatively, 1-(ethylsulfonyl)azetidin-3-one (4) is prepared by dissolving (3) and an oxammonium catalyst, for example TEMPO, 4-AA TEMPO, 4-hydryoxy TEMPO, AZADO or 3 carbamoyl-PROXYL in an appropriate solvent, preferably EtOAc. The substrate to catalyst ratio may be between 1:1 and 50,000:1. It may be possible to use a substrate to catalyst ratio greater than 50,000:1 but catalyst handling may become a limiting factor. The preferred range of substrate to catalyst ratio is 500:1 to 10,000:1. The preferred substrate to catalyst ratio is 1000:1. The substrate/catalyst solution is added to a suspension of TCCA and sodium acetate in an appropriate solvent, such as EtOAc. After substrate feed is complete, the reaction is stirred for an appropriate amount of time until the reaction is complete. The reaction product may be isolated by techniques known to the skilled artisan or taken to the next reaction without isolation. Preferably, the solids are removed by filtration and the organic layer is concentrated to an oil, which is displaced with IPA to deliver compound (4). The IPA solution of (4) may be used directly in the synthesis of (1).

[1-(Ethylsulfonyl)azetidin-3-ylidene]acetonitrile(1) is prepared utilizing Horner-Wadsworth-Ernmons conditions by combining a slight excess of an appropriate phosphonate reagent, such as diethyl cyanomethylphosphonate, and 1-(ethylsulfonyl)azetidin-3-one (4) in an appropriate alcoholic solvent, preferably IPA. The resulting solution is cooled to a temperature colder than room temperature, preferably 0° C., and an appropriate base, such as DIPEA, is added while the temperature is maintained at a temperature colder than room temperature, preferably 0 to 5° C. Typically, the mixture is stirred for 1 to 5 hours. Upon completion of the reaction as monitored by standard techniques, the reaction is optionally seeded with [1-(ethylsulfonyl)azetidin-3-ylidene]acetonitrile and an appropriate anti-solvent, preferably heptane, is added. The reaction product is isolated by techniques known to the skilled artisan. Optionally, the product (1) may be further purified by a seeded recrystallization in an appropriate alcoholic solvent, such as IPA or water or a mixture thereof.

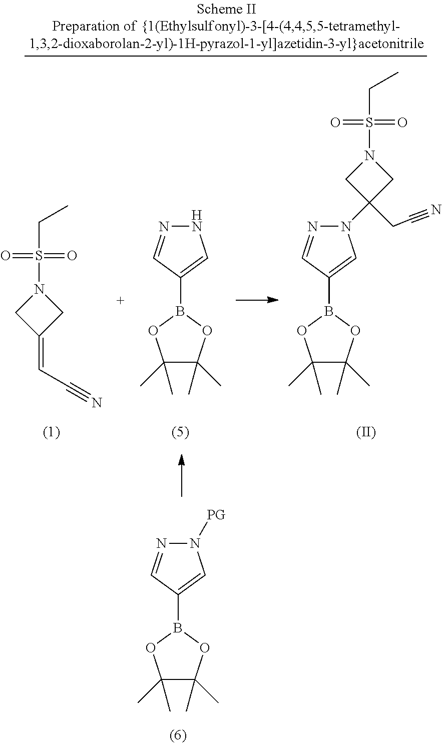

PG is an appropriate nitrogen protecting group. 4-(4,4,5,5-Tetramethyl-1,3,2-dioxaborolan-2-yl)-1H-pyrazole (5) may be obtained by deprotecting the corresponding compound (6) using appropriate conditions to effect the removal of the protecting group. See for example “Greene's Protective Groups in Organic Synthesis”, Fourth Edition, by Peter G. M. Wuts and Theodora W. Greene, John Wiley and Sons, Inc. 2007. {1-(Ethylsulfonyl)-3-[4-(4,4,5,5-tetramethyl-1,3,2-dioxaborolan-2-yl)-1H-pyrazol-1-yl]azetidin-3-yl}acetonitrile (II) is prepared by coupling equimolar equivalents of 2-(1-ethylsulfonylazetidin-3-ylidene)acetonindle (1) and 4-(4,4,5,5-tetramethyl-1,3,2-dioxaborolan-2-yl)-1H-pyrazole (5) in the presence of a catalytic amount of DBU, potassium tert-butoxide, tetramethylguanidine (TMG), or tert-butyl tetramethylguanidine (t-BuTMG) can also be used. Preferably, t-BuTMG is used. A slight excess of either (1) or (5) may be used. Suitable solvents include DMF, CPME, ACN, THF, and 2-MeTHF. The preferred solvent system is THF/CPME. Catalytic amounts of t-BuTMG are added to the reaction mixture. Preferably 0.04-0.10 equivalents of t-BuTMG are added. The reaction temperature may be maintained at approximately room temperature or heated above room temperature. Preferably, the reaction temperature should be maintained at temperatures between 20 to 70° C. The reaction is maintained until conversion of the starting materials (1) and (5) to the product, {1-(ethylsulfonyl)-3-[4-(4,4,5,5-tetramethyl-1,3,2-dioxaborolan-2-yl)-1H-pyrazol-1-yl]azetidin-3-yl}acetonitrile (II), is completed as evidenced by monitoring techniques known to the skilled artisan.

After the reaction is complete, a suitable solvent to effect crystallization, such as 1-propanol or CPME or a mixture thereof, is added to the reaction mixture optionally followed by seed crystals of {1-(ethylsulfonyl)-3-[4-(4,4,5,5-tetramethyl-1,3,2-dioxaborolan-2-yl)-1H-pyrazol-1-yl]azetidin-3-yl}acetonitrile (II). The reaction mixture may be optionally cooled to a temperature below room temperature, preferably about 0° C. before the crystallization step is commenced. Further, the temperature may be held at a temperature below room temperature with optional stirring for 0 to 24 hours once crystallization commences. The resulting solids are collected by standard procedures, preferably by filtration or centrifugation, and subsequently washed with appropriate solvents, such as 1-propanol, heptane, CPME, or a mixture of said solvents. Optionally, the solid product (II) collected may be dried by standard techniques.

Alternatively, 4-(4,4,5,5-tetramethyl-1,3,2-dioxaborolan-2-yl)-1H-pyrazole (5) may be prepared by reacting equimolar equivalents of 1-(1-ethoxyethyl)-4-(4,4,5,5-tetramethyl-1,3,2-dioxaborolan-2-yl)-1H-pyrazole (6) in a suitable solvent such as CPME, ACN, toluene, and 2-MeTHF, preferably CPME at a temperature of 0 to 34° C., in the presence of an acid, such as anyhydrous HCl, acetyl chloride in methanol, and sulfuric acid. Preferably the acid is anhydrous HCl. A scavenger, such as 2,3-dimethylbutane-2,3-diol, for the byproduct of the deprotection reaction may be added since the reaction is an equilibrium process which is driven by the removal of the by product. After the addition of the acid, the reaction temperature may be optionally warmed to about room temperature. The completion of the reaction is monitored by standard monitoring techniques. Typically, the reaction is complete after 1 to 6 hours. The product (5) may be isolated by standard techniques or may be carried directly on to the next reaction.

Alternatively, one equivalent of 4-(4,4,5,5-tetramethyl-1,3,2-dioxaborolan-2-yl)-1H-pyrazole (5) is combined with about 1.5 equivalents of [1-(ethylsulfonyl)azetidin-3-ylidene]acetonitrile (1) and heated to above room temperature, preferably about 50° C. (solution A). Concurrently, an equivalent of 4-(4,4,5,5-tetramethyl-1,3,2-dioxaborolan-2-yl)-1H-pyrazole (5) is combined with a catalytic amount of 2-tert-butyl-1,1,3,3-tetramethylguanidine (about 0.16 equiv.) in an appropriate solvent such as CPME and THF, THF, 2-MeTHF, or acetonitrile, preferably CPME, and heated to above room temperature, preferably 65 to 70° C. (solution B). The temperature of solution A is maintained above room temperature, preferably about 50 to 65° C. Solution A is added to Solution B. Once addition is complete, the reaction is heated, preferably to about 65 to 70° C., and monitored for completion by standard techniques, such as HPLC, LC/MS, or TLC. Typically, the reaction is complete within one to five hours. A suitable solvent to effect crystallization is added and the solution is cooled. Preferably, the solvent is 1-propanol. The reaction is preferably cooled to about 5 to 55° C. Optionally, a solvent exchange via distillation may be employed to change the solvents from CPME/THF to n-propanol. The product may then be crystallized from n-propanol. Optionally, seed crystals may be added. The resulting solid is collected by standard techniques known to the skilled artisan.

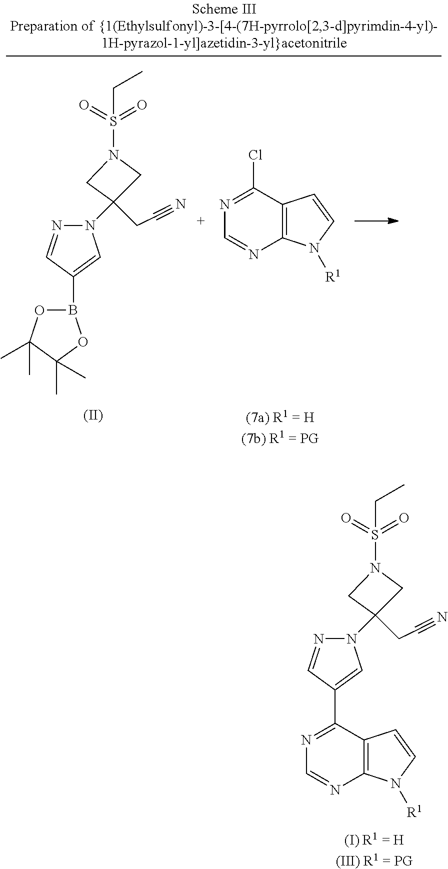

For compounds (7b) and (III), PG is a nitrogen protecting group, such as tert-butoxycarbonyl.

Compound (7b), tert-butyl 4-chloropyrrolo[2,3-d]pyrimidine-7-carboxylate, may be prepared utilizing a biphasic technique wherein 4-chloro-7H-pyrrolo[2,3-d]pyrimidine (7a) in an appropriate solvent, preferably THF or methyltetrahydrofuran, is added to a solution of water and a base, preferably tripotassium phosphate, also known as potassium phosphate tribasic or K₃PO₄, which has been cooled to an approximate temperature slightly below room temperature. The temperature of the reaction is preferably 20-25° C. Typically, the mixture is stirred for 1 to 10 hours. Upon completion of the reaction as monitored by standard techniques, the aqueous phase is removed. The compound may be isolated by standard techniques or taken on without further purification.

Compound (III), tert-butyl 4-{1-[3-(cyanomethyl)-1-(ethylsulfonyl)azetidin-3-yl]-1H-pyrazol-4-yl}-7H-pyrrolo[2,3-d]pyrimidine-7-carboxylate, may be prepared by standard palladium coupling conditions, preferably Suzuki-Miyaura conditions, by reacting equimolar amounts of 2-[1-(ethylsulfonyl-3-[4-(4,4,5,5-tetramethyl-1,3.2-dioxaborolan-2-yl)pyrazol-1-yl]azetidin-3-yl]acetonitrile (II) and tert-butyl 4-chloropyrrolo[2,3-d]pyrimidine-7-carboxylate (7b) in the presence of a slight excess of di-tert-butyl dicarbonate in THF and a catalytic amount of a Pd(II) reagent, preferably, dichloro[1,1’-bis(dicyclohexylphosphino)ferrocene] palladium(H) or PdCl₂-XantPhos, under an inert atmosphere of preferably nitrogen gas or argon gas. The skilled artisan will appreciate that the Suzuki-Miyaura reaction may be effected using a number of suitable palladium reagents. Such suitable reagents are described in Chem. Rev. 2011, 111, 1417-1492. Preferably the reaction is performed in a biphasic solution. A solution of aqueous potassium phosphate is added. The reaction temperature is heated to temperatures above room temperature. Preferably, the reaction temperature is maintained at 50-75° C. Typically, the mixture is stirred for 1 to 10 hours. Upon completion of the reaction as monitored by standard techniques, the reaction temperature is cooled slightly, preferably by 10° C., and a non-polar solvent, preferably hexanes is added to effect precipitation of the product. The resulting suspension is stirred for an additional 1 to 4 hours and then cooled to room temperature or slightly below, preferably 20-25° C. The solid is collected by standard techniques known to the skilled artisan. The reagents may be added all at once or added sequentially.

Alternatively, 4-chloro-7H-pyrrolo[2,3-d]pyrimidine (7a) and THF is added to a solution of about two equivalents of di-tert-butyl dicarbonate and a catalytic amount of potassium tert-butoxide in a suitable solvent, preferably THF, and cooled to a temperature at or slightly below room temperature, preferably 20-25 ‘C. A slightly cooled aqueous solution of potassium phosphate tribasic is added followed by {1-(ethylsulfonyl)-3-[4-(4,4,5,5-tetramethyl-1,3,2-dioxaborolan-2-yl)-1H-pyrazol-1-yl]azetidin-3-yl}acetonitrile (II). A suitable Pd(II) catalyst, preferably dichloro[1,1′-bis(dicyclohexylphosphino)ferrocene] palladium(II) or PdCl₂-XantPhos, is added. The reaction is heated to a temperature above room temperature, preferably 55 to 60° C. Typically, the reaction is complete after 4 hours. The aqueous phase is removed. Compound (III) is isolated by techniques known to one skilled in the art.

The transformation of compound (III) to compound (I) may be effected by thermal cleavage. For example, a solution of compound (III) in an appropriate solvent such as THF, aqueous THF, butanol, or aqueous butanol, preferably aqueous THF, is stirred at room temperature or heated at 50 to 100° C. to give a solution of compound (I) in THF. The solution may be kept at room temperature or heated to a temperature above room temperature. Also, the solution may be at atmospheric pressure or at higher pressure. Compound (I) may be optionally purified and/or optionally crystallized.

Alternatively, {1-(ethylsulfonyl)-3-[4-(7H-pyrrolo[2,3-d]pyrimdin-4-yl)-1H-pyrazol-1-yl]azetidin-3-yl}acetonitrile (I) may be synthesized without isolating tert-butyl 4-{1-[3-(cyanomethyl)-1-(ethylsulfonyl)azetidin-3-yl]-4yl}-7H-pyrazol-4-yl) -7H-pyrrolo[2,3-d]pyrimidine-7-carboxylate (III). For example, under an inert atmosphere, preferably of argon or nitrogen, equimolar amounts of 4-chloro-7H-pyrrolo[2,3-d]pyrimidine (7a), {1-(ethylsulfonyl)-3-[4-(4,4,5,5-tetramethyl-1,3,2-dioxaborolan-2-yl)-1H-pyrazol-1-yl]azetidin-3-yl}acetonitrile (II), and potassium phosphate tribasic are added to a catalytic amount of a mixture of a Pd(II) catalyst, preferably dichloro[1,1′-bis(dicyclohexylphosphino)ferrocene] palladium(II), with potassium phosphate tribasic in an appropriate solvent system, preferably THF and water in a 4:1 ratio. The reaction may be heated to a temperature above room temperature. Typically, the reaction is stirred for 1 to 24 hours. After completion of the reaction, optionally monitored by standard techniques known to the skilled artisan, the reaction mixture is cooled and the resulting product is isolated by techniques known to the skilled artisan.

The following preparations and examples further illustrate the invention. Unless noted to the contrary, the compounds illustrated herein are named and numbered using Accelrys® Draw version 4.1 (Accelrys, Inc., San Diego, Calif.) or IUPACNAME ACDLABS.

### Preparation 1

### 1-(Ethylsulfonyl)azetidin-3-ol

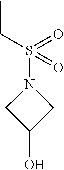

Water (210 mL), potassium phosphate tribasic (63.9 g, 301 mmol) and sodium hydroxide (11 g, 273.8 mmol) are added together and stirred until dissolution is observed. The basic solution is cooled to 20° C. and azetidine-3-ol hydrochloride (30 g, 273.8 mmol), water (30 mL), and THF (150 mL) are added. The bi-phasic mixture is vigorously stirred and a solution of ethanesulfonyl chloride (35.2 g, 273.8 mmol) dissolved in THF (60 mL) is added at a consistent rate over at least 2 hours while holding the reaction temperature at 20° C. The reaction mixture is stirred for 1 hour after the addition is complete. The organic layer is removed by distillation, resulting in about 360 g aqueous solution.

### Batch Extraction:

The aqueous solution is extracted with toluene (3×90 mL) to remove 1-(ethylsulfonyl)azetidin-3-yl ethanesulfonate and the combined organic extracts are discarded, The aqueous layer is extracted with EtOAc (3×90 ml). The organic extracts are combined and concentrated to about 90 mL volume. EtOAc (180 mL) is added and the mixture is concentrated to about 90 mL volume to give the title compound (about 85% yield by GC). The crude solution is used directly without further purification. ¹H NMR (400 MHz, d₆-DMSO) δ 5.78 (s, 1H), 4.39 (d, J=4.4 Hz, 1H), 3.90 (dd, J=6.8, 8.6 Hz, 2H), 3.67-3.63 (m, 2H), 3.06 (q, J=7.3 Hz, 2H), 1.19 (t, J=7.3 Hz, 3H).

### Continuous Counter Current Extraction:

The continuous workup is scaled based on a 1 kg azetidin-3-ol reaction. The continuous extraction setup uses four 250 ml flasks “mixers” with high mixing velocity, peristaltic pumps to transfer solutions to the settlers and gravity to feed back to flasks or product flasks.
Toluene extraction: Feed process with 4.42 mL/minute toluene into mixer 1 and 12.58 mL/minute aqueous crude solution of 1-(ethylsulfonyl)azetidin-3-ol into mixer 4, resulting in about 9.4 minutes of mixing in each mixer for a total of about 37 minutes of mixing time total and a total of about 52 minutes of time in the system. The toluene solution is discarded. The aqueous solution containing 1-(ethylsulfonyl)azetidin-3-ol is further processed to extract 1-(ethylsulfonyl)azetidin-3-ol.
EtOAc Extraction: Feed process with 4.8 mL/minute EtOAc into mixer 1 and 11.7 mL/minute aqueous solution containing 1-(ethylsulfonyl)azetidin-3-ol into mixer 4, resulting in about 9.7 minutes of mixing in each mixer for a total of about 39 minutes of mixing time total and a total of about 53 minutes of time in the system. The extracted aqueous solution is discarded and the EtOAc solution containing 1-(ethylsulfonyl)azetidin-3-ol is concentrated at 35° C. under vacuum to a light yellow oil to give the title compound (about 95%). ¹H NMR (400 MHz, d₆-DMSO) δ 5.78 (s, 1H), 4.39 (d, J=4.4 Hz, 1H), 3.90 (dd, J=6.8, 8.6 Hz, 2H), 3.67-3.63 (m, 2H), 3.06 (q, J=7.3 Hz, 2H), 1.19 (t, J=7.3 Hz, 3H).

### Alternate Preparation 1a

Water (70 g), potassium phosphate tribasic (21.3 g, 100 mmol) and sodium hydroxide (3.65 g; 91.3 mmol) are added together and the mixture is stirred until dissolution is observed. The basic solution is cooled to 20° C. and azetidin-3-ol hydrochloride (10 g, 91.3 mmol) and THF (50 mL) are added. The bi-phasic mixture is vigorously stirred and a solution of ethanesulfonyl chloride (11.7 g, 91.3 mmol) diluted with THF (20 mL) is added at a consistent rate over at least 2 hours while maintaining the reaction temperature at 20° C. The reaction mixture is stirred for 1 hour after the addition is complete. The organic layer is removed by distillation, resulting in about a 112 g aqueous solution. The aqueous solution is extracted with toluene (3×30 mL) and the combined organic extracts are discarded. The aqueous layer is extracted with EtOAc (3×30 mL). The organic extracts are combined and concentrated to about 30 mL volume. EtOAc (60 mL) is added and the mixture is concentrated to about 30 mL volume. EtOAc (60 mL) is again added and the mixture is concentrated to about 30 mL volume. The final solution assayed by GC to reveal 85% in situ yield of the title compound with a total water content <0.2 weight %. The crude solution is used directly without further purification. ¹H NMR (400 MHz, d₆-DMSO) δ 5.78 (s, 1H), 4.39 (d, J=4.4 Hz, 1H), 3.90 (dd, J=6.8, 8.6 Hz, 2H), 3.67-3.63 (m, 2H), 3.06 (q, J=7.3 Hz, 2H), 1.19 (t, J=7.3 Hz, 3H).

### Preparation 2

### 1-(Ethylsulfonyl)azetidin-3-one

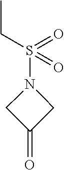

### Batch:

TEMPO (1.55 g, 9.92 mmol) is dissolved in acetonitrile (70 mL). Sodium nitrite (0.68 g, 9.86 mmol) is dissolved in water (35 mL) in a second vessel. In a third vessel is added 1-(ethylsulfonyl)azetidin-3-ol (35 g, 196.6 mmol), acetic acid (11.37 mL), and acetonitrile (70 mL). The solutions are combined in a sealed vessel, the headspace is filled with 6% O₂ in N₂, and the system is pressurized to 3447.38 kPa with this gas mixture. The system is set to stir at 350 rpm. The reactor headspace is purged and replaced with 6% O₂ in N₂ every 60 seconds during the reaction, using an automated control system. The reaction is run for 17 hours with headspace cycling. GC assay shows 30.75 g, 95.9% in situ yield. The reaction is then split in two, and half of the product mixture is worked up as follows: Starting with 125.27 g of the reaction mixture, containing a theoretical 23.75 g product, the mixture is neutralized to pH 7.02 with DIPEA (15.06 g). Water is added to dissolve the D1PEAHCl salt and the mixture is extracted with 90/10 EtOAc/heptane (5×125 mL). The organic extracts are combined and concentrated to approximately 105 mL and IPA (525 mL) is added. The mixture is concentrated to approximately 105 mL and further IPA (525 mL) is added. This process is repeated 3 x and after the final concentration, IPA (70 mL) is added to provide a solution of the title product (22.32 g, 93.9%) in 175 IPA.

### Flow:

Feed solutions for the continuous flow aerobic oxidation are prepared in glass pressure bottles with pressure transfer heads. Feed 1: TEMPO (1.54 g, 9.86 mmol) is charged to a pressure bottle and dissolved in acetonitrile (35 mL). Feed 2: Sodium nitrite (0.68 g, 9.86 mmol) is added to a pressure bottle and dissolved in water (35 mL). Feed 3: 1-(ethylsulfonyl)azetidin-3-ol is added to a third pressure bottle (35 g, 196.6 mmol) with acetic acid (11.28 mL) and acetonitrile (70 mL). The feeds are charged to the feed pumps: Feed 1 is charged to feed pump A, Feed 2 is charged to feed pump C and Feed 3 is charged to feed pump B. The pumps are started: pump A is started at 0.0123 mL/minute, pump B is started at 0.036 mL/minute and pump C is started at 0.0116 mL/minute. For these runs, 6.259% O₂ in N₂ is used; the targeted flow rate of gas is 5.791 mmol/minute, and the back end pressure is maintained at 3447.38 kPa. The reaction is continued for 12 hours to give an in situ yield of 98%. Not all of the material is collected from this continuous run but a representative amount of material is collected during the latter part of the run and worked up as follows The solution is diluted with water (30 mL) and the solution is neutralized to pH 7 by adding DIPEA. The mixture is extracted with 90/10 EtOAc/heptane (5×100 mL). The organic extracts are combined, concentrated to approximately 60 mL and IPA (300 mL) is added, the mixture is concentrated to approximately 60 mL and IPA (300 mL) is added. This process is repeated 3 x and after the final concentration, IPA (40 mL) is added to provide a solution of the title product (19.48 g, 87.4%) in IPA (100 mL).

### Alternate Preparation 2A

1-(Ethylsulfonyl)azetidin-3-ol (40.6 g, 246 mmol), TEMPO (38.4 mg. 0.246 mmol) and EtOAc (257 mL) are combined and the mixture is stirred for 2 hours. In a separate vessel, TCCA (28.7 g 123 mmol), sodium acetate (26.3 g, 321 mmol) and EtOAc (171 mL) are added and the mixture is cooled to <3° C. under a N₂ atmosphere. The 1-(ethylsulfonyl)azetidin-3-ol solution is added (10 mL). After the initial exotherm subsides, the remaining solution is added while maintaining a vessel temperature of <11° C. for about 1.5 hours. The substrate/catalyst feed vessel is rinsed with EtOAc (25 mL) and the mixture is stirred an additional 2 hours. IPA (16.2 g, 270 mmol) is added, the mixture is warmed to 10° C., and stirred 18 hours. Powdered K₂CO₃ (34.1 g, 247 mmol) is added and the mixture is stirred an additional 4 hours. The inorganic salts are removed by filtration and the waste filter cake is washed with EtOAc (160 mL). The waste cake is washed with additional EtOAc (120 mL). The combined filtrate (about 650 mL) is concentrated to about 200 mL volume with a maximum jacket temperature of 40° C. IPA (350 mL) is added and the mixture is concentrated to about 200 mL volume. IPA (350 mL) is added again and the mixture is concentrated to about 200 mL volume. The final solution is tested for water (<0.2%) and EtOAc (<1%) and the title product (99.5% yield by GC) is used directly without further purification. ¹H NMR (400 MHz, d₆-DMSO) δ 4.84 (s, 4H), 3.28 (q, J=7.3 Hz, 2H), 1.26 (t, J=7.5 Hz, 3H).

### Alternate Preparation 2B

From a similar preparation as described in la, a crude solution of 1-(ethylsulfonyl)azetidin-3-ol (50 g, 287.5 mmol) in EtOAc (30 mL), 3-carbamoyl-PROXYL (50 mg. 0.27 mmol) and EtOAc (200 mL) are added together and the mixture is stirred for 2 hours. In a separate vessel, TCCA (33.6 g 143 mmol), sodium acetate (30.8 g, 375 mmol) and EtOAc (300 mL) are added together and the mixture is cooled to <3° C. under a N₂ atmosphere. The 1-(ethylsulfonyl)azetidin-3-ol solution is added (12 mL) and then the remaining solution is added while maintaining a vessel temperature <10° C. for about 1.5 hours. The substrate/catalyst feed vessel is rinsed with EtOAc (25 mL) and the mixture is stirred an additional 2 hours. IPA (24 mL) is added, the mixture is warmed to 10 “C, and stirred 16 hours. Powdered K₂CO₃ (40.3 g, 292 mmol) is added and the mixture is stirred for 4 hours. The inorganic salts are removed by filtration and the waste filter cake is washed with EtOAc (300 mL). The combined filtrate (about 650 mL) is concentrated to about 200 mL volume with a maximum jacket temperature of 40° C. IPA (350 mL) is added and the mixture is concentrated to about 200 mL volume. IPA (350 mL) is added and the mixture is concentrated again to about 200 mL volume. The final solution is tested for water (<0.2%) and EtOAc (<1%) and the title product (185 g of solution with 43.6 g title compound, 93%) is used directly without further purification. ¹H NMR (400 MHz, d₆-DMSO) δ 4.84 (s, 4H), 3.28 (q, 7.3 Hz, 21I),1.26 (t, J=7.5 Hz, 3H).

### Alternate Preparation 2C

1-(Ethylsulfonyl)azetidin-3-ol (75 g, 431 mmol), 4-hydroxyTEMPO (75 mg. 0.435 mmol) and EtOAc (300 mL) are combined and the mixture is stirred for 1 hour. In a separate vessel, TCCA (50.1 g 216 mmol), sodium acetate (46.1 g, 562 mmol) and EtOAc (450 mL) are added and the mixture is cooled to <3° C. under a N₂ atmosphere. The 1-(ethylsulfonyl)azetidin-3-ol solution (20 mL) is added. After the initial exotherm subsides, the remaining solution is added while maintaining a vessel temperature <6° C. for about 1.5 hours. The substrate/catalyst feed vessel is rinsed with EtOAc (25 mL) and the reaction mixture is stirred an additional 1 hour. IPA (33 mL, 432 mmol) is added, the mixture is warmed to 10° C., and stirred 2 hours. Powdered K₂CO₃ (60.0 g, 434 mmol) is added and the mixture is stirred an additional 20 hours. The inorganic salts are removed by filtration and the waste filter cake is washed with EtOAc (600 mL). The combined filtrate (about 1300 mL) is concentrated to an oil using a maximum jacket temperature of 40° C. IPA (200 mL) is added and the mixture is re-concentrated to an oil (73.7g, 91.4% potency, 95.6% yield). The oil is used without further purification. ¹H NMR (400 MHz, d₆-DMSO) δ 4.84 (s, 4H), 3.28 (q, J=7.3 Hz, 21I), 1.26 (t, J=7.5 Hz, 3H).

### Alternate Preparation 2D

1-(Ethylsulfonyl)azetidin-3-ol (75 g, 431 mmol), 4-acetamidoTEMPO (86 mg. 0.429 mmol) and EtOAc (300 mL) are combined and the mixture is stirred for 1 hour. In a separate vessel, TCCA (50.1 g 216 mmol), sodium acetate (46.1 g, 562 mmol) and EtOAc (450 mL) are added and the mixture is cooled to <3° C. under a N₂ atmosphere. The 1-(ethylsulfonyl)azetidin-3-ol solution (20 mL) is added. After the initial exotherm subsides the remaining solution is added while maintaining a vessel temperature of <6° C. for about 1.5 hours. The substrate/catalyst feed vessel is rinsed with EtOAc (25 mL) and the reaction mixture is stirred an additional 1 hour. IPA (33 mL) is added, the mixture is warmed to 10° C., and stirred 2 hours. Powdered K₂CO₃ (60.0 g, 434 mmol) is added and the mixture is stirred an additional 20 hours. The inorganic salts are removed by filtration and the waste filter cake is washed with EtOAc (600 mL). The combined filtrate (about 1300 mL) is concentrated to an oil using a maximum jacket temperature of 40° C. IPA (200 mL) is added and the mixture is concentrated to an oil (75.6 g, 92.3% potency, 99.1%). The oil is used without further purification. ¹H NMR (400 MHz, d₆-DMSO) δ 4.84 (s, 4H), 3.28 (q, J=7.3 Hz, 2H), 1.26 (t, J=7.5 Hz, 3H).

### Alternate Preparation 2E

1-(Ethylsulfonyl)azetidin-3-ol (75 g, 95% purity, 431 mmol), 2-azaadamantane N-oxyl (86 mg. 0.561 mmol) and EtOAc (300 mL) are combined and the mixture is stirred for 1 hour. In a separate vessel, TCCA (50.1 g 216 mmol), sodium acetate (46.1 g, 562 mmol) and EtOAc (450 mL) are added and the mixture is cooled to <3° C. under a N₂ atmosphere. The 1-(ethylsulfonyl)azetidin-3-ol solution (20 mL) is added. After the initial exotherm subsides the remaining solution is added while maintaining a vessel temperature of <6° C. for about 1.5 hours. The substrate/catalyst feed vessel is rinsed with EtOAc (25 mL) and the reaction mixture is stirred an additional 1 hour. IPA (33 mL, 432 mmol) is added, the mixture is warmed to 10 ‘V, and stirred 2 hours. Powdered K₂CO₃ (60.0 g, 434 mmol) is added and the mixture is stirred an additional 20 hours. The inorganic salts are removed by filtration and the waste filter cake is washed with EtOAc (600 mL). The combined filtrate (about 1300 mL) is concentrated to an oil using a maximum jacket temperature of 40° C. IPA (200 mL) is added and the mixture is concentrated to an oil (75.7 g, 94.4% potency, 101.5%). The oil is used without further purification. ¹H NMR (400 MHz, d₆-DMSO) δ 4.84 (s, 4H), 3.28 (q, J=7.3 Hz, 2H), 1.26 (t, J=7.5 Hz, 3H).

### Preparation 3

### [1-(Ethylsulfonyl)azetidin-3-ylidene]acetonitrile

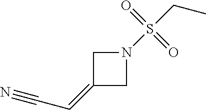

Diethyl cyanomethylphosphonate (48.6 g, 274 mmol) is added to an IPA solution (225 mL) of 1-(ethylsulfonyl)azetidin-3-one (41 g, 251 mmol). The resulting solution is cooled to 0° C. and DIPEA (44.2 g, 348 mmol) is added at a rate such that the temperature is maintained at 5° C. The mixture is stirred for 1 hour and seeded with [1-(ethylsulfonyl)azetidin-3-ylidene]acetonitrile. The mixture is stirred an additional 3 hours maintaining the temperature at 0-5° C. and then warmed to 10° C. and stirred an additional 16 hours. The suspension is cooled to 0° C. then heptane (225 mL) is added over at least an hour, and the mixture is stirred for an additional hour at 0° C. The reaction mixture is filtered and the resulting precipitate is rinsed with 1:2 IPA/heptane (120 mL) and dried at 30° C. for at least 12 hours to give the title compound (41 g, 83.4%) as a white powder. The material (30 g, 159 mmol) is purified by a seeded recrystallization in an IPA (120 mL)/water (12 mixture to give the title compound (28.7 g, 90.7%). Melting point=68° C., ES/MS m/z 187.0527 [M+H]⁺; ¹H NMR (400 MHz, d₆-DMSO) δ 5.89 (quintet, J=2.5 Hz, 1H), 4.76 (q, J=3.1 Hz, 2H), 4.67 (dd, J=2.6, 5.7 Hz, 2H), 3.21 (q, J=7.3 Hz, 2H), 1.21 (t, J=7.3 Hz, 3H), ¹³C NMR (500 MHz, d6-DMSO) δ 7.3, 42.5, 58.7, 59.1, 94.0, 115.0, 156.3.

### Preparation 4

### {1-(Ethylsulfonyl)-3-[4-(4,4,5,5-tetramethyl-1,3,2-dioxaborolan-2-yl)-1H-pyrazol-1-yl]azetidin-3-yl) acetonitrile

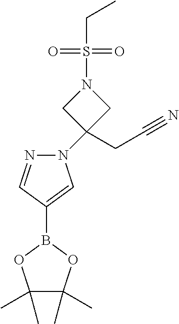 [SMILES: CCS(=O)(=O)N1CC(CC#N)(n2cc(B3OC(C)(C)C(C)(C)O3)cn2)C1]

4-(4,4,5,5-Tetramethyl-1,3,2-dioxaborolan-2-yl)-1H-pyrazole (15.22 g, 77.44 mmol) and 2-(1-ethylsulfonylazetidin-3-ylidene)acetonitrile (14.42 g, 77.43 mmol) are combined in DMF (45.6 mL). After the solids have dissolved, DBU (0.50 g, 3.28 mmol) is added. The solution is stirred at 20-25° C. for 22 hours and then concentrated to a thick oil under vacuum at 65° C. 1-Propanol (150 mL) is added followed by seed crystals (30 mg) of the title compound. The product crystallizes and the resulting slurry is stirred for 1.75 hours. The solids are collected by filtration, washed with 1-propanol (20 mL) followed by heptane (20 mL), then dried to give the title compound (24.7 g, 82.8%). ¹H NMR (d₆-DMSO) δ 1.20 (t, 3H), 1.25 (s, 12H), 3.18 (q, 2H), 3.58 (s, 2H), 4.13 (d, 2H), 4.43 (d, 2H), 7.57 (s, 1H), 8.34 (s 1H).

### Alternate Preparation 4a

1-(1-Ethoxyethyl)-4-(4,4,5,5-tetramethyl-1,3,2-dioxaborolan-2-yl)-1H-pyrazole (60.00 g, 224 mmol) is combined with CPME (120 mL) and 2,3-dimethylbutane-2,3-diol (26.49 g, 224 mmol). The reaction is cooled to 5-10° C. then a solution of anhydrous HCl in CPME (3.1 M, 86.8 mL, 269 mmol) is added over 15 minutes, followed by additional CPME (15 mL). The reaction is stirred at 20-25° C. and monitored for completion. After 7 hours, additional HCl solution (3 mL, 9.3 mmol) is added to the reaction and stirring is continued for an additional 15 hours to give 4-(4,4,5,5-tetramethyl-1,3,2-dioxaborolan-2-yl)-1H-pyrazole hydrochloride salt, which is not isolated. The reaction mixture is cooled to about 0° C. then a solution of triethylamine (30.8 g, 304 mmol) in CPME (21 mL) is added over 10 minutes. The reaction mixture temperature increases to 20° C. following the addition. Additional CPME (10 mL) is added and the reaction mixture is stirred for 15 minutes then cooled in an ice bath. After 3 hours the reaction mixture is filtered and the solid (triethylamine hydrochloride) is washed with cold CPME (3×60 mL). The filtrate and washes are combined to give 426 g of solution containing 102.3 mg of 4-(4,4,5,5-tetramethyl-1,3,2-dioxaborolan-2-yl)-1H-pyrazole/g solution (total of 45.57 g, 100% yield of 4-(4,4,5,5-tetramethyl-1,3,2-dioxaborolan-2-yl)-1H-pyrazole) which is used directly in the next step. A portion of the solution (298.8 g of solution containing 30.5 g, 157.2 mmol of 4-(4,4,5,5-tetramethyl-1,3,2-dioxaborolan-2-yl)-1H-pyrazole) is combined with [1-(ethylsulfonyl)azetidin-3-ylidene]acetonitrile (27.90 g, 147 mmol) and CPME (46 mL). To this solution is added a solution of DBU (2.33 g, 14.7 mmol) in CPME (45 mL). The reaction mixture is heated to 70° C. and monitored for completion. The reaction is stirred for 16 hours then 1-propanol (40 mL) is added. The solution is cooled to approximately 54° C. and seed crystals (2.85 g) of the title compound are added. The resulting slurry is cooled to 0° C. over 6 hours and held at that temperature for 14 hours. The solids are collected by filtration, washed with cold 9:1 v/v CPME/1-propanol (3×57 mL) then dried to give the title compound (48.6 g, 81.8%).

### Alternate Preparation 4b

1-(1-Ethoxyethyl)-4-(4,4,5,5-tetramethyl-1,3,2-dioxaborolan-2-yl)-1H-pyrazole (12.0 g, 44.73 mmol) is combined with CPME (20 mL) and 2,3-dimethylbutane-2,3-diol (5.9 g, 49.20 mmol) and the residual solid is rinsed into the reaction vessel with CPME (4 mL). The solution is cooled to about 10° C. and a solution of anhydrous HCl in CPME (3.0 M, 18.6 mL, 55.91 mmol) is added over 5 minutes. The reaction is warmed to 25° C. and monitored for completion. After 4 hours at 25 ‘C, additional HCl solution (3.0 M, 5 mL, 15.03 mmol) is added to the reaction and stirring is continued for an additional 1.5 hours to give 4-(4,4,5,5-tetramethyl-1,3,2-dioxaborolan-2-yl)-1H-pyrazole hydrochloride salt which is not isolated. The reaction mixture is cooled to about 10° C. and a solution of triethylamine (6.3 g, 62.17 mmol) in CPME (8 mL) is added over 7 minutes. The reaction mixture temperature increases to about 20° C. after the addition. The resulting slurry is stirred at 25° C. for 16 hours. The reaction mixture is cooled to about 0° C. for 1.5 hours and filtered. The solids (triethylamine hydrochloride) are washed with cold CPME (2×12 mL) to give 78.70 g of solution containing 103.6 mg of 4-(4,4,5,5-tetramethyl-1,3,2-dioxaborolan-2-yl)-1H-pyrazole/g solution, (total of 8.15 g, 94% yield of 4-(4,4,5,5-tetramethyl-1,3,2-dioxaborolan-2-yl)-1H-pyrazole) which is used directly in the next step. A portion of the solution (35.34 g, 40.4 mL, 18.94 mmol of 4-(4,4,5,5-tetramethyl-1,3,2-dioxaborolan-2-yl)-1H-pyrazole) is combined with [1-(ethylsulfonyl)azetidin-3-ylidene]acetonitrile (6.00 g, 31.57 mmol) then warmed to 50° C. to effect a solution (solution A). Concurrently, another portion of the solution (35.34 g, 40.4 mL, 18.94 mmol of 4-(4,4,5,5-tetramethyl-1,3,2-dioxaborolan-2-yl)-1H-pyrazole) is combined with 2-tert-butyl-1,1,3,3-tetramethylguanidine (0.55 g, 3.16 mmol), rinsed in with CPME (3 mL) and heated to 65-70° C. (solution B). Solution A is kept at about 50° C. and drop wise added to solution B. The addition vessel is rinsed with CPME (6 mL). The reaction is stirred at 70° C. and monitored for completion (typical completion time is two hours). 1-Propanol (8.3 mL) is added and the solution is cooled to approximately 55° C. Seed crystals (0.6 g) of the title compound are added and the resulting slurry is held at a temperature of 55° C. for 1 hour then cooled to −3° C. over 9 hours and held at that temperature for at least 2 hours. The solids are collected by filtration, washed with cold 9:1 v/v CPME/1-propanol (2×12 mL) and dried to give the title compound (10.30 g, 85.8%).

### Alternate Preparation 4c

Preparation of 4-(4,4,5,5-tetramethyl-1,3,2-dioxaborolan-2-yl)-1H-pyrazole solution (107.0 mg/g as a solution in CPME) is conducted using the previously described procedure denoted in Alternate Preparation 5b.

The solution of 4-(4,4,5,5-tetramethyl-1,3,2-dioxaborolan-2-yl)-1H-pyrazole (69.41 g, 79.8 mL, 38.27 mmol) is combined with 2-tert-butyl-1,1,3,3-tetramethylguanidine (0.54 g, 3.15 mmol) followed by a CPME rinse (6 mL) then heated to about 65° C. 2-(1-(Ethylsulfonylazetidin-3-ylidene)acetonitrile (6.00 g, 31.90 mmol) is dissolved in THF (14 mL) and added over 3 hours followed by a THF rinse (2 mL). The reaction is stirred at about 65° C. and monitored for completion (typical completion time is two hours post-addition). The solution is cooled to 25° C. and concentrated to a wet residue. 1-Propanol (60 mL) is added and the suspension is again concentrated to a wet solid. The solid is suspended in 1-propanol (90 mL) and heated to 67° C. to form a solution. The solution is cooled to 57° C. and seed crystals (0.6 g) of the title compound are added. The resulting slurry is held at a temperature of 57° C. for 2 hours then cooled to −3° C. over 9 hours and held at that temperature for at least 2 hours. The solids are collected by filtration, washed with cold 1-propanol (2×12 mL) and dried to give the title compound (10.89 g, 89.8%).

### Preparation 5

### tert-Butyl 4-chloropyrrolo[2,3-d]pyrimidine-7-carboxylate

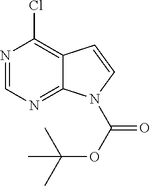 [SMILES: CC(C)(C)OC(=O)n1ccc2c(Cl)ncnc21]

In an autoclave is combined potassium phosphate tribasic (414.6 g, 1.95 mol) and water (520 mL). The solution is cooled to 20-25° C. then 4-chloro-7H-pyrrolo[2,3-d]pyrimidine (100.0 g, 651.2 mmol) and 2-methyltetrahydrofuran (2.1 L) are added. After 7 hours the aqueous phase is removed. The organic phase is washed with water (2×300 mL.) and concentrated to a white solid. The solid is combined with heptane (400 mL) and stirred at 20-25° C. to give a suspension. The suspension is cooled to 0° C. for 2 hours and the product is isolated by filtration. The isolated solids are washed with cold heptane (200 mL) and dried under vacuum to give the title compound (141.5 g, 86%) as a white solid. ¹H NMR (d₆-DMSO) δ 1.61 9s, 9H), 6.80 (d, 1H), 7.94 (d, 1H), 8.80 (s, 1H)

### Preparation 6

### tert-Butyl 4-{1-[3-(cyanomethyl)-1-(ethylsulfonyl)azetidin-3-yl]-1H-pyrazol-4-yl}-7H-pyrrolo[2,3-d]pyrimidine-7-carboxylate

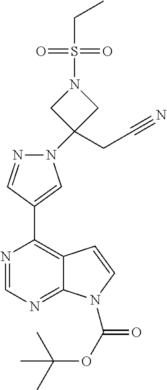 [SMILES: CCS(=O)(=O)N1CC(CC#N)(n2cc(-c3ncnc4c3ccn4C(=O)OC(C)(C)C)cn2)C1]

To an autoclave under nitrogen is added 2-[1-(ethylsulfonyl-3-[4-(4,4,5,5-tetramethyl-1,3,2-dioxaborolan-2-yppyrazol-1-yl]azetidin-3-yl]acetonitrile (750 mg, 1.97 mmol), tert-butyl 4-chloropyrrolo[2,3-d]pyrimidine-7-carboxylate (500 mg, 1.97 mmol) and a solution of di-tert-butyl dicarbonate in THF (0.29 M, 6.8 mL, 1.98 mmol) to form a solution. To this solution is added a 10 mg/mL solution of dichloro[1,1′-bis(dicyclohexylphosphino)ferrocene] palladium(II) in dichloromethane (0.15 mL, 0.0020 mmol). A solution of aqueous potassium phosphate (3.13 M, 1.90 mL, 5.95 mmol) and water (3.8 mL) is charged to the autoclave and the mixture is heated to 60° C. After 4 hours the aqueous phase is removed, the temperature is adjusted to 50° C. and hexanes (6.8 mL) are added to effect a precipitate. The suspension is stirred for two hours at 50° C. then cooled to 20-25° C. The solids are collected by filtration and washed with 1:1 v/v THF/hexanes (5 mL) and dried under vacuum to give the title compound (0.83 g, 89%). ¹H NMR (dₒ-DMSO) δ 1.23 (t, 3H), 1.62 (s, 9H), 3.22 (q, 2H), 3.68 (s, 2H), 4.23 (d, 2H), 4.59 (d, 2H), 7.31 (d, 1H), 7.91 (d, 1H), 8.49 (s, 1H), 8.90 (s, 1H), 8.97 (s, 1H)

### Alternate Preparation 6a

### tert-Butyl 4-{1-[3-(cyanomethyl)-1-(ethylsulfonyl)azetidin-3-yl]-1H-pyrazol-4-yl}-7H-pyrrolo[2,3-d]pyrimidine-7-carboxylate

4-Chloro-7H-pyrrolo[2,3-d]pyrimidine (6.0 g, 39.07 mmol), potassium phosphate tribasic (24.88 g, 117.21 mmol), di-cert-butyl dicarbonate (17.05 g, 78.12 mmol), THF (126 mL), and water (31.2 mL) are combined and stirred at 20-25° C. under an oxygen-free atmosphere for 17 hours to give tert-butyl 4-chloropyrrolo[2,3-d]pyrimidine-7-carboxylate (9.91 g, 39.07 mmol) as a biphasic solution. To the biphasic solution is added dichloro[1,1′-bis(dicyclohexylphosphino)ferrocene] palladium(II) (300 mg, 0.397 mmol) and 2-[1-(ethylsulfonyl)-3-[4-(4,4,5,5-tetramethyl-1,3,2-dioxaborolan-2-yl)pyrazol-1-yl]azetidin-3-yl]acetonitrile (14.84 g, 39.02 mmol). The reaction is heated at 60° C. under an oxygen-free atmosphere with vigorous agitation for 9 hours while monitoring for completion. The layers are allowed to separate and the aqueous layer is removed. The remaining organic layer is treated with a silica-thiol resin (13.8 g) and the mixture is stirred for 14 hours at 60° C. The resin is removed by filtration and washed with hot THF (20 mL) to provide a solution of tert-butyl 4-{1-[3-(cyanomethyl)-1-(ethylsulfonyl)azetidin-3-yl]-1H-pyrazol-4-yl}-7H-pyrrolo [2,3-d]pyrimidine-7-carboxylate. This solution is concentrated to dryness under reduced pressure, and combined with 1-butanol (81 mL) and water (24 mL). The resulting suspension is heated to 90° C. and the solution is stirred at that temperature for 5 hours. The solution is allowed to cool to 20-25° C. over 2 hours and stirred for an additional 2 hours at 20-25° C. The crystals are collected by filtration, washed with 1-butanol (40 mL), and dried to give the title compound (13.04 g, 89.9%). A portion of the solids (12.06 g) are recrystallized from 4:1 (v/v) 1-butanol/water (78 mL) to give the title compound (11.56 g, 95.9%) as a white solid with potency of about 100%.

### EXAMPLE 1

### {1-(Ethylsulfonyl)-3-[4-(7H-pyrrolo [2,3-d]pyrimdin-4-yl)-1H-pyrazol-1-yl]azetidin-3-yl}acetonitrile

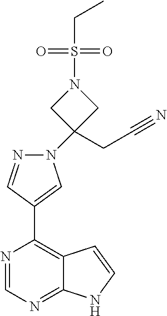 [SMILES: CCS(=O)(=O)N1CC(CC#N)(n2cc(-c3ncnc4[nH]ccc34)cn2)C1]

To an autoclave under nitrogen is added 4-chloro-7H-pyrrolo[2,3-d]pyrimidine (2.202 g, 13.15 mmol), {1-(ethylsulfonyl)-3-[4-(4,4,5,5-tetramethyl-1,3.2-dioxaborolan-2-yl)-1H-pyrazol-1-yl]azetidin-3-yl}acetonitrile (5.00 g, 13.15 mmol), potassium phosphate tribasic (2.80 g, 13.19 mmol) and a mixture of dichloro[1,1′-bis(dicyclohexylphosphino)ferrocene] palladium(II) with potassium phosphate tribasic (2.84 g of the mixture containing 50 mg total with 0.07 mmol palladium catalyst). THF (21 mL) is added to the autoclave followed by water (5.3 mL). The autoclave is sealed and the contents are heated to 90° C. for 19 hours. The reaction mixture is cooled and the resulting suspension is diluted with THF (40 mL) and water (10 mL). The solution is filtered through a mixture of diatomaceous earth (0.4 g) and carbon (0.2 g). The filtrate is concentrated under vacuum to remove THF. An aqueous buffer solution (pH=7, 30 mL) is added followed by 1-butanol (30 mL). The mixture is heated to 85° C. with stirring to dissolve residual solids. Stirring is stopped and the lower aqueous layer is removed. Water (10 mL) is added to the stirred 1-butanol layer. Stirring is discontinued and the lower aqueous layer is removed. The 1-butanol layer is cooled to 75° C. and stirred for 30 minutes. The solution is further cooled to 20° C. over 6 hours and the resulting slurry is held at that temperature overnight. The solids are collected by filtration, washed with 9:1 v/v 1-butanol/water (10 mL) and dried at 40° C. to give the title compound (3.45 g, 70.6%). The title compound (2.5 g, 6.73 mmol) is combined with 1-butanol (12.6 mL) and water (3.8 mL). The mixture is heated to 85° C. and stirred for 30 minutes. The solution is cooled to 20° C. over 7 hours to provide a slurry. The solids are collected by filtration then washed with 1-butanol followed by water. The solids are dried to give the title compound (2.25 g, 90% after recrystallization of 2.5 g).

### Alternate Preparation, Example 1b

To an autoclave under nitrogen is added di-tert-butyl Bicarbonate (118.1 g, 540.9 mmol) and THF (415 mL). After the solids have dissolved a 1 M solution of potassium tert-butoxide in THF (13.6 mL, 13.6 mmol) is added to the autoclave and the mixture is heated to 55-60° C. In a separate flask under nitrogen is combined 4-chloro-7H-pyrrolo[2,3-d]pyrimidine (41.51 g, 270.3 mmol) and THF (603 mL). After the solids have dissolved, the solution of 4-chloro-7H-pyrrolo[2,3-d]pyrimidine is added over 1 hour to the autoclave containing the di-tert-butyl dicarbonate/potassium tert-butoxide solution. Once the 4-chloro-7H-pyrrolo[2,3-d]pyrimidine addition is complete the autoclave is cooled to 20-25° C. and the system is purged of carbon dioxide. In a separate flask under nitrogen, potassium phosphate tribasic (172.1 g, 810.7 mmol) is combined with water (360.6 mL). The solution of potassium phosphate is cooled to 20-25° C. then added to the autoclave. After the potassium phosphate solution has been added to the autoclave, {1-(ethylsulfonyl)-3-[4-(4,4,5,5-tetramethyl-1,3,2-dioxaborolan-2-yl)-1H-pyrazol-1-yl]azetidin-3-yl}acetonitrile (105.8 g, 278.3 mmol) is added to the autoclave as a solid and the autoclave is purged of any oxygen. In a separate flask under nitrogen, PdCl₂-XantPhos (0.506 g, 0.669 mmol) is combined with THF (103.5 ml.) and water (10.4 mL) to provide a yellow solution. The solution of PdCl₂-XantPhos is then added to the autoclave and the mixture is heated to 55 to 60° C. After 4 hours the aqueous phase is removed from the autoclave. In a separate flask under nitrogen, sodium chloride (14.23 g, 243.5 mmol) and water (266 mL) are combined to form a solution. The sodium chloride solution is added to the autoclave and the mixture is stirred for 30 minutes. The aqueous phase is then removed from the autoclave and the remaining contents are cooled to 20 to 25° C. to give a solution containing the intermediate tert-butyl 4-chloro-7H-pyrrolo[2,3-d]pyrimidine-7-carboxylate in THF/water (1395 mL, 1328.8 g, 9.28 wt % of intermediate tert-butyl 4-chloro-7H-pyrrolo[2,3-d]pyrimidine-7-carboxylate, 97%).

The above solution is passed through a column containing a silica thiol resin at 60° C. to aid in removal of the palladium followed by a thermal cleavage (140° C.) of the Boc protecting group under pressure (2068.43 kPa) to give a solution of the title compound in THF. The columns are flushed with an 88:12 wt/wt THF/water to give a 7.06 wt % solution of the title compound in THF/water.

To an autoclave under nitrogen is added the above title compound solution in THF/water (592.0 mL, 550.6 g solution, 104.6 mmol). The solution is concentrated by atmospheric distillation to a volume of 140 mL. 1-Butanol (432 mL) is charged to the autoclave and the mixture is cooled to 25-30° C. The pressure is reduced to 75 mm Hg and the mixture is concentrated under vacuum to a volume of 264 mL. Water (80.5 mL) is added to the autoclave and the temperature is adjusted to 95° C. to effect a solution. The solution is cooled to 84° C. then seeded with the title compound (2.4 g, 6.6 mmol). After cooling to ambient temperature the product is isolated by filtration. The solids are rinsed with a solution of 10:1 v/v 1-butanol/water (2×77 mL) then dried under vacuum to give the title compound (35.1 g, 90%). ¹H NMR (d₆-DMSO) δ 1.23 (t, 3H), 3.22 (q, 2H), 3.68 (s, 2H), 4.22 (d, 2H), 4.59 (d, 2H), 7.07 (d, 1H), 7.61 (d, 1H), 8.46 (s, HI), 8.69 (s, 1H), 8.92 (s, 1H), 12.12 (s, 1H)

### Alternate Preparation Example 1c

tert-Butyl 4-{1-[3-(cyanomethyl)-1-(ethylsulfonyl)azetidin-3-yl]-1H-pyrazol-4-yl}-7H-pyrrolo[2,3-d]pyrimidine-7-carboxylate (2.0 g, 4.2 mmol) is suspended in a mixture of 1-butanol (13.0 mL) and water (4.0 ml), and heated to 90° C. with stirring. The resulting light yellow solution is stirred at 90° C. until less than 1% starting material remains by HPLC analysis (typically about 4 hours). The solution is allowed to slowly cool to 20-25° C. over several hours. After two more hours at room temperature, the solid is collected by vacuum filtration, washed with 1-butanol (5 ml), and dried in vacuo at 40° C. overnight to provide the title compound as a white solid (1.46 g 92.7%).

1. A process for the preparation of {1-(ethylsulfonyl)-3-[4-(7H-pyrrolo[2,3-d]pyrimidin-4-yl)-1H-pyrazol-1-yl]azetidin-3-yl} acetonitrile (I) comprising the steps of:
i) Coupling azetidine-3-ol hydrochloride (2) with ethanesulfonyl chloride to give 1-ethylsulfonylazetidin-3-ol (3);
ii) Aerobic oxidation of 1-ethylsulfonylazetidin-3-ol (3) to 1-(ethylsulfonyl)azetidin-3-one (4) in the presence of a nitroxyl reagent, an oxidizing reagent, and an acid under an oxygen atmosphere; or oxidation of 1-ethylsulfonylazetidin-3-ol (3) to 1-(ethylsulfonyl)azetidin-3-one (4) with TCCA and a catalytic oxammonium reagent;
iii) Reaction of 1-(ethylsulfonyl)azetidin-3-one (4) with a phosphonate reagent in the presence of a base to prepare compound (1);
iv) Optionally crystallizing [1-(ethylsulfonyl)azetidin-3-ylidene]acetonitrile (1);
v) Optionally protecting 4-(4,4,5,5-tetramethyl-1,3,2-dioxaborolan-2-yl)-1H-pyrazole (5) with a nitrogen protecting group;
vi) Coupling [1-(ethylsulfonyl)azetidin-3-ylidene]acetonitrile (1) and 4-(4,4,5,5-tetramethyl-1,3,2-dioxaborolan-2-yl)-1H-pyrazole (5) in the presence of a non-nucleophilic base to give {1-(ethylsulfonyl)-3-[4-(4,4,5,5-tetramethyl-1,3,2-dioxaborolan-2-yl)-1H-pyrazol-1-yl]azetidin-3-yl}acetonitrile (II);
vii) Optionally crystallizing {1-(ethylsulfonyl)-3-[4-(4,4,5,5-tetramethyl-1,3,2-dioxaborolan-2-yl)-1H-pyrazol-1-yl]azetidin-3-yl}acetonitrile (II);
viii) Optionally protecting 4-chloro-7H-pyrrolo[2,3-d]pyrimidine (7a) with a nitrogen protecting group;
ix) Coupling {1-(ethylsulfonyl)-3-[4-(4,4,5,5-tetramethyl-1,3,2-dioxaborolan-2-yl)-1H-pyrazol-1-yl]azetidin-3-yl}acetonitrile (II) with 4-chloro-7H-pyrrolo[2,3-d]pyrimidine (7a) or tert-butyl 4-chloropyrrolo[2,3-d]pyrimidine-7-carboxylate (7b) using a Pd(II) catalyst in the presence of a base to provide { 1-(ethylsulfonyl)-3-[4-(7H-pyrrolo[2,3-d]pyrimidin-4-yl)-1H-pyrazol-1-yl]azetidin-3-yl}acetonitrile (I) or tert-butyl 4-{1-[3-(cyanomethyl)-1-(ethylsulfonyl)azetidin-3-yl] -1H-pyrazol-4-yl}-7H-pyrrolo[2,3-d] pyrimidine-7-carboxylate (III);
x) Optionally deprotecting tert-butyl 4-{1-[3-(cyanomethyl)-1-(ethylsulfonyl)azetidin-3-yl]-1H-pyrazol-4-yl}-7H-pyrrolo[2,3-d]pyrimidine-7-carboxylate (III) to {1-(ethylsulfonyl)-3-[4-(7H-pyrrolo [2,3-d]pyrimidin-4-yl)-1H-pyrazol-1-yl]azetidin-3-yl}acetonitrile (I); and
xi) Optionally crystallizing {1-(ethylsulfonyl)-3-[4-(7H-pyrrolo[2,3-d]pyrimidin-4-yl)-1H-pyrazol-1-yl]azetidin-3-yl}acetonitrile (I).

2. A process of claim 1 comprising the steps of:
i) Optionally protecting 4-chloro-7H-pyrrolo[2,3-d]pyrimidine (7a) with a nitrogen protecting group;
ii) Coupling {1-(ethylsulfonyl)-3-[4-(4,4,5,5-tetramethyl-1,3,2-dioxaborolan-2-yl)-1H-pyrazol-1-yl]azetidin-3-yl}acetonitrile (II) with 4-chloro-7H-pyrrolo[2,3-d]pyrimidine (7a) or tert-butyl 4-chloropyrrolo[2,3-d]pyrimidine-7-carboxylate (7b) using a Pd(II) catalyst in the presence of a base to provide {1-(ethylsulfonyl)-3-[4-(7H-pyrrolo[2,3-d]pyrimidin-4-yl)-1H-pyrazol-1-yl]azetidin-3-yl}acetonitrile (I) or tert-butyl 4-{1-[3-(cyanomethyl)-1-(ethylsulfonyl)azetidin-3-yl]-1H-pyrazol-4-yl}-7H-pyrrolo[2,3-d]pyrimidine-7-carboxylate (III);
iii) Optionally deprotecting tert-butyl 4-{1-[3-(cyanomethyl)-1-(ethylsulfonyl)azetidin-3-yl]-1H-pyrazol-4-yl}-7H-pyrrolo[2,3-d]pyrimidine-7-carboxylate (III) to {1-(ethylsulfonyl)-3-[4-(7H-pyrrolo[2,3-d]pyrimidin-4-yl)-1H-pyrazol-1-yl]azetidin-3-yl}acetonitrile (I); and
iv) Optionally crystallizing {1-(ethylsulfonyl)-3-[4-(7H-pyrrolo[2,3-d]pyrimidin-4-yl)-1H-pyrazol-1-yl]azetidin-3-yl}acetonitrile (I).

3. A process of claim 2 wherein the Pd(II) catalyst is dichloro[1,1′-bis(dicyclohexylphosphino)ferrocene] palladium(II) or (9,9-dimethyl-9H-xanthene-4,5-diyl)bis(diphenylphosphane) dichloropalladium.

4. A process of claim 2 wherein the base is K₃PO₄ or potassium tert-butoxide.

5. A process of claim 1 for the preparation of 2-[1-ethylsulfonyl-3-[4-(4,4,5,5-tetramethyl-1,3,2-dioxaborolan-2-yl)pyrazol-1-yl]azetidin-3-yl]acetonitrile (II) comprising the steps of:
i) Coupling [1-(ethylsulfonyl)azetidin-3-ylidene]acetonitrile (1) and 4-(4,4,5,5-tetramethyl-1,3,2-dioxaborolan-2-yl)-1H-pyrazole (5) in the presence of a non-nucleophilic base; and
ii) Optionally crystallizing 2-[1-ethylsulfonyl-3-[4-(4,4,5,5-tetramethyl-1,3,2-dioxaborolan-2-yl)pyrazol-1-yl]azetidin-3-yl]acetonitrile (II).

6. A process of claim 5 wherein the non-nucleophilic base is 1,8-diazabicyclo[5.4.0]undec-7-ene, 2-tert-butyl-1,1,3,3-tetramethylguanidine, potassium tert-butoxide, or tetramethylguanidine.

7. A process of claim 1 for the preparation of [1-(ethylsulfonyl)azetidin-3-ylidene]acetonitrile (1) comprising the steps of:
i) Coupling azetidine-3-ol hydrochloride (2) with ethanesulfonyl chloride to give 1-ethylsulfonylazetidin-3-ol (3);
ii) Aerobic oxidation of the alcohol of 1-ethylsulfonylazetidin-3-ol (3) to 1-(ethylsulfonyl)azetidin-3-one (4) in the presence of a nitroxyl reagent, an oxidizing reagent, and an acid under an oxygen atmosphere;
iii) Reaction of 1-(ethylsulfonyl)azetidin-3-one (4) with phosphonate reagent in the presence of a base to prepare compound (1); and
iv) Optionally crystallizing [1-(ethylsulfonyl)azetidin-3-ylidene]acetonitrile (1).

8. A process of claim 7 wherein the nitroxyl reagent is 2,2,6,6-tetramethyl-1-piperidinyloxy free radical.

9. A process of claim 7 wherein the oxidizing reagent is NaNO₃.

10. A process of claim 7 wherein the oxygen atmosphere is 5-8% O₂ in N₂.

11. A process of claim 12 wherein the oxygen atmosphere is 6% O₂ in N₂.

12. A process of claim 7 wherein the phosphonate reagent is diethyl cyanomethylphosphonate.

13. A process of claim 7 wherein the base is diisopropylethylamine.

14. A process of claim 1 for the preparation of [1-(ethylsulfonyl)azetidin-3-ylidene]acetonitrile (1) comprising the steps of:
i) Coupling azetidine-3-ol hydrochloride (2) with ethanesulfonyl chloride to give 1-ethylsulfonylazetidin-3-ol (3);
ii) Oxidation of 1-ethylsulfonylazetidin-3-ol (3) to 1-(ethylsulfonyl)azetidin-3-one (4) with TCCA and a catalytic oxammonium reagent;
iii) Reaction of 1-(ethylsulfonyl)azetidin-3-one (4) with phosphonate reagent in the presence of a base to prepare compound (1); and
iv) Optionally crystallizing [1-(ethylsulfonyl)azetidin-3-ylidene]acetonitrile (1).

15. A process of claim 14 wherein the catalytic oxammonium reagent is TEMPO.

16. A process of claim 1 wherein the reactions are performed using flow reaction methodology.

17. A process of claim 1 wherein the reactions are performed using batch processing methodology.

18. A crystalline form of the compound, 2-[1-ethylsulfonyl-3-[4-(4,4,5,5-tetramethyl-1,3,2-dioxaborolan-2-yl)pyrazol-1-yl]azetidin-3-yl]acetonitrile:

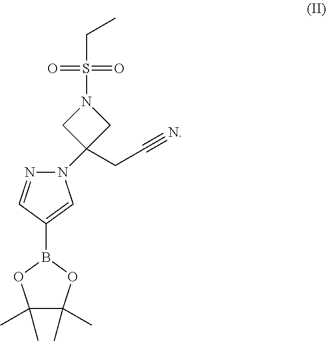 [SMILES: CCS(=O)(=O)N1CC(CC#N)(n2cc(B3OC(C)(C)C(C)(C)O3)cn2)C1]

19. (canceled)
